(19) United States
(12) Patent Application Publication
(10) Pub. No.: US 2023/0263552 A1
(43) Pub. Date: Aug. 24, 2023

(54) METHODS AND DEVICES FOR AUTOMATED MICROFLUIDIC OOCYTE DENUDATION

(71) Applicant: CORNELL UNIVERSITY, Ithaca, NY (US)

(72) Inventors: Amir MOKHTARE, Ithaca, NY (US); Gianpiero D. PALERMO, New York, NY (US); Alireza ABBASPOURRAD, Ithaca, NY (US)

(21) Appl. No.: 18/014,537

(22) PCT Filed: Jul. 6, 2021
(86) PCT No.: PCT/US2021/040507

§ 371 Date: Jan. 5, 2023

Related U.S. Application Data
(60) Provisional application No. 63/048,531, filed on Jul. 6, 2020.

(51) Int. Cl. A61B 17/435; C12M 3/06; C12M 3/00; C12M 1/42; C12M 1/00

(52) U.S. Cl. CPC A61B 17/435; C12M 23/16; C12M 21/06; C12M 35/04; C12M 29/14; A61D 19/04

(57) ABSTRACT

The present invention relates to a microfluidic device for denudation of a cumulus oocyte complex. The device includes a substrate. A first channel having a width of about 200 μm to about 1 mm is located within the substrate. The first channel extends from a first end to a second end of the substrate. The first channel has a one or more ridge elements located along a surface thereof. A first port is located in the substrate and in fluid communication with the first end of the channel. A second port is located in the substrate and in fluid communication with the second end of the channel. Systems and methods of use of the microfluidic device for denudation of a cumulus oocyte complex are also disclosed.

This application claims the priority benefit of U.S. Provisional Patent Application Ser. No. 63/048,531, filed Jul. 6, 2020, which is hereby incorporated by reference in its entirety.

### FIELD

The present technology relates to methods and devices for automated microfluidic oocyte denudation.

### BACKGROUND

Oocyte denudation involves the removal of cumulus cells from a cumulus oocyte complex (COC) to produce a denuded oocyte. Oocyte denudation is a prerequisite step of embryonic cell micromanipulation. Clinical procedures that are the prevailing human infertility treatments, such as intracytoplasmic sperm injection (ICSI), as discussed in Palermo et al., “Pregnancies after Intracytoplasmic Injection of Single Spermatozoon into an Oocyte,” Lancet 340:17-18 (1992), and in vitro fertilization, as discussed in Rock et al., “In Vitro Fertilization and Cleavage of Human Ovarian Eggs,” Science 100:105-107 (1944), require adequate oocyte denudation and cumulus cell removal for their success. In addition, mammalian embryo research advances such as transgenic and cloned animal production, as discussed in Wilmut et al., “Viable Offspring Derived from Fetal and Adult Mammalian Cells,” Nature 385:810-813 (1997) and Wakayama et al., “Full-Term Development of Mice from Enucleated Oocytes Injected with Cumulus Cell Nuclei,” Nature 394:369-374 (1998), as well as emergent CRISPR based genetic engineering methods, as discussed in Wang et al., “One-Step Generation of Mice Carrying Mutations in Multiple Genes by CRISPR/Cas-Mediated Genome Engineering,” Cell 153:910-918 (2013), all depend on the crucial preparation of denuded oocytes.

Cumulus and corona layer removal allows unequivocal assessment of oocyte cytoplasmic and extracytoplasmic status. Visualization of the first polar body that resides in the previtalane space and presence of mitotic spindle as indicative of nuclear maturity is achieved only after completion of the oocyte denudation step. Complete and meticulous cumulus removal also reduces the risk of sperm DNA contamination with the extraneous DNA from maternal cumulus cells when a polymerase chain reaction (PCR) based technique is employed, as discussed in Rienzi et al., “Oocyte Denuding. In: Nagy Z P, Varghese A C, Agarwal A, Editors. In Vitro Fertilization: A Textbook of Current and Emerging Methods and Devices. Cham: Springer International Publishing; 2019. pp. 133-145. Partial removal of cumulus can compromise the accuracy of micromanipulation by preventing adequate holding of oocyte during microinjection. Furthermore, byproducts of the cumulus/hyaluronidase reaction can result in zona pellucida breakdown and reduced embryo viability if cumulus cells are left intact. This method is associated with risks, such as oocyte parthenogenetic activation, PB1 and Miotic spindle dislocation, zona pellucida fracture, and oocyte degeneration

Cumulus cells are removed prior to any micromanipulation procedure by either mechanical or a combination of chemical and mechanical treatment. Mechanical treatment involves using a set of appropriately sized glass/plastic pipettes, and repeated flushing of the COCs through each pipet vigorously. Mechanical aspiration starts in a pipette with larger diameter and ends with a pipette with an internal diameter slightly smaller than the oocyte diameter.

However, in the most common and established method, cumulus cells are first enzymatically treated with hyaluronidase (HA) to loosen the hyaluronan-based bonds in the surrounding COCs matrix followed by mechanical treatment. The HA alone does not disperse the cumulus cells and mainly is used to aid the mechanical pipetting described above.

Both methods are usually performed within wells and small oil covered micro droplets on a warm plate under a stereomicroscope. Painstaking care and effort should be taken during the manual pipetting as it inevitably imposes some mechanical stress on oocytes. Although mechanical pipetting alone is immune from enzymatic complications, it forces COCs into a narrow micro capillary and imposes high mechanical stress on them, which can ultimately result in zona pellucida fracture and oocyte degeneration.

Enzymatic treatment can reduce the mechanical stress on oocytes at the cost of adding further steps to the denudation procedure. On the other hand, it has been shown that high doses of HA combined with vigorous retrieval and pipetting can induce parthenogenetic activation oocytes in mice and humans, as discussed in Palermo et al., “Sperm Characteristics and Outcome of Human Assisted Fertilization by Subzonal Insemination and Intracytoplasmic Sperm Injection,” Fertil Steril 59:826-835 (1993), and Muechler et al., “Parthenogenesis of Human Oocytes as a Function of Vacuum Pressure,” J In Vitro Fert Embryo Transf 6:335-337 (1989). Likewise, but less frequently, vigorous pipetting can dislocate the polar body and expose the mitotic spindle to the microneedle during the microinjection. Numerous transfers and washing steps also expose the COCs to temperature, pH, and oxygen level variations that can impair oocyte developmental potential. Additionally, the manual denudation procedure requires a highly skilled embryologist and suffers from inter-intra operator variability. Therefore, it would be advantageous to develop an automated system to not only alleviate the mechanical stresses, minimize COC transfer and handling steps, but also to reduce the labor while providing a consistent and optimal denudation procedure and easy tracking of individual cells.

Other practitioners have proposed solutions, but challenges still exist. For example, Zeringue et al., “Removal of Cumulus from Mammalian Zygotes using Microfluidic Techniques,” BiomedMicrodevices 3:219-224 (2001) described a microfluidic-based device for mechanical removal of the cumulus cells from individual COCs. The device consists of a main channel with dimensions on the same order of the COCs. Each COC is first squeezed in two regions with slightly narrower diameter than the ovum to reshape the COC mass into a doughnut shape. The cumulus cells are removed in a corner with two narrow ports by suction and then flushed backed into the loading container. The device requires manual control of multiple fluid flows and processes one oocyte at a time. The oocytes are also susceptible to pinching at the denudation ports that can impose high and irreversible mechanical stresses on them. The same technique was also applied in in vitro fertilization (IVF) to remove cumulus from zygote stage cells. Such studies showed that microfluidic based cumulus removal increases the development potential of the early mammalian embryo and enhances the efficiency of the procedure.

In another example, Weng et al., “On-Chip Oocyte Denudation from Cumulus-Oocyte Complexes for Assisted Reproductive Therapy,” Lab Chip.18:3892-3902 (2018), demonstrated an on chip oocyte denudation device for COC denudation. The device consisted of a microchannel with repeated jagged surface expansion-contraction units. The enzymatically treated COCs were loaded into a microfluidic tube and connected to a syringe pump. Continuous fluid flow pushed COCs through the jagged conduits and progressively got narrower toward the end of the channels. In this device COCs are pushed against jagged walls with physical contact, which can result in their excessive deformation. Due to similar dimensions to the COC itself and constant microchannel height, the device is also susceptible to clogging. The loading of the cell to a tube also increases the risk of cell loss and unwanted mechanical stress.

Han et al., “Cumulus Removal And Single Mammal-Ian Oocyte Trapping on a Microfluidic Device,” rsc.org. Available: https://www.rsc.org/binaries/LOC/2009/Pdf/621-W61F.pdf, described a hybrid vacuum and gravity based device for cumulus removal. The device consisted of an inlet and two outlet ports for loading and waste removal and oocyte entrapment. By applying a negative pressure to the waste outlet, COCs submerged in HA solution were brought to a digesting region, when cumulus cells are dispersed and approach the junction of the two outlets, the negative pressure is stopped and oocytes are allowed to enter the storage micro chamber by gravity. The device loading ports and cylinders included flat ends that may lead to cell loss in the inlet port. The device also was based on gravity, which is comparable with adhesion and capillary forces in that scale. Thus, there is an ongoing and unmet need for new or improved methods and devices for oocyte denudation.

The present invention is directed to overcoming these and other deficiencies in the art.

### SUMMARY

One aspect of the present technology relates to a microfluidic device for denudation of a cumulus oocyte complex. The device includes a substrate. A first channel having a width of about 200 μm to about 1 mm is located within the substrate. The first channel extends from a first end to a second end of the substrate. The first channel has one or more ridge elements located along a surface thereof. A first port is located in the substrate and in fluid communication with the first end of the channel. A second port is located in the substrate and in fluid communication with the second end of the channel.

Another aspect of the present technology relates to a system for denudation of a cumulus oocyte complex. The system includes the microfluidic device of the present technology. An optical imaging device is configured to image a portion of the channel including a cumulus oocyte complex of the microfluidic device. A computing device is coupled to the optical imaging device. The computing device includes a processor coupled to a memory and configured to execute programmed instructions stored in the memory including determining, based on one or more images received from the optical imaging device, a state of denudation of the cumulus oocyte complex located in the portion of the channel. One or more instructions are provided to the controller to alternately open and close the first valve and the second valve.

Yet another aspect of the present technology relates to a method for denudation of a cumulus oocyte complex. The method includes providing the microfluidic device of the present technology. A fluid including a cumulus oocyte complex is introduced into the channel of the microfluidic device through the first port. The first valve and the second valve are activated such that the cumulus oocyte complex is translated along the channel in a first direction toward the second end from the first end along the one or more ridge elements.

A further aspect of the present technology relates to a method for denudation of a cumulus oocyte complex. The method includes providing the system of the present technology. A fluid including a cumulus oocyte complex is introduced into the channel of the microfluidic device through the first port. The first valve and the second valve are activated such that the cumulus oocyte complex is translated along the channel in a first direction toward the second end from the first end along the one or more ridge elements. The position and/or state of the cumulus oocyte complex is monitored using the optical imaging device. The activation of the first valve and the second valve is adjusted based on the position and/or state of the cumulus oocyte complex.

The disclosed devices and methods advantageously provide an integrated, semi-automated device to perform the denudation procedure for a cumulus oocyte complex (COC). The disclosed devices and methods achieve the automation of the denudation process with a substantial decrease in imposed mechanical stress through a non-contact denudation technique. The device is also highly flexible, such that the procedure can be adjusted based on the chemical treatment, cell variability, and denudation efficiency. The devices and methods also provide for standardization of the procedure to minimize the variability associated with manual operation. The device may also be constructed of inexpensive components and simple equipment that can be further integrated with other modules to improve the affordability and efficiency of assisted reproductive therapies.

The disclosed devices and methods minimize physical stresses on the oocyte, provide for automated COC handling and transfer, complete cumulus cell depletion, and easy visual tracking of an individual oocyte. The disclosed devices and methods overcome previous challenges by generating transverse or secondary flows in a microchannel by placing ridges, for example, on the roof of the channel at an oblique angle, ∅, with respect to the longitudinal axis of the microchannel. These ridges induce an anisotropic resistance to laminar flows. An axial pressure gradient generates a mean transverse component in the flow that originates at the structured surface and circulates back across the bottom of the channel, forming helical streamlines.

### BRIEF DESCRIPTION OF THE DRAWINGS

FIG. 1 is a partial block diagram and partial schematic of an environment including a first embodiment of a microfluidic device of the present technology.

FIG. 2A is a perspective phantom view of a microfluidic chip of the microfluidic device of the present technology.

FIG. 2B is an enhanced view of the first port of the microfluidic chip shown in FIG. 2A.

FIG. 2C is an enhanced view of the channel of the microfluidic chip shown in FIG. 2A.

FIG. 2D is an enhanced view of the second port of the microfluidic chip shown in FIG. 2A.

FIG. 3A is a perspective view of the channel of the microfluidic chip shown in FIG. 2A.

FIG. 3B is a schematic view of the channel of the microfluidic chip shown in FIG. 2A.

FIG. 4A is a perspective view of another exemplary channel with chevron ridge elements that may be employed in the present technology.

FIG. 4B is a schematic view of the exemplary channel shown in FIG. 4A.

FIG. 5 is a block diagram of the exemplary computing device illustrated in FIG. 1.

FIG. 6 is a partial block diagram and partial schematic of an environment including a second embodiment of a microfluidic device of the present technology.

FIG. 7A is a perspective phantom view of another exemplary microfluidic chip of the present technology.

FIG. 7B is an enhanced view of a full valve on a supplementary channel of the microfluidic chip shown in FIG. 7A.

FIG. 7C is an enhanced view of a sieve valve on the channel of the microfluidic chip shown in FIG. 7A.

FIG. 8A is a top view of the microfluidic chip shown in FIG. 7A.

FIG. 8B is an enhanced view of a sieve valve shown in FIG. 8A.

FIGS. 9A and 9B are side cross-sectional views of a portion of the microfluidic chip shown in FIG. 7A with a sieve valve before pressurization (FIG. 9A) and after pressurization (FIG. 9B).

FIGS. 9C and 9D are side cross-sectional views of a portion of the microfluidic chip shown in FIG. 7A with a full valve before pressurization (FIG. 9C) and after pressurization (FIG. 9C).

FIG. 10A shows a cohort of oocytes retrieved from super-ovulated B6D2F1 adult mice.

FIG. 10B shows a MII stage mouse oocyte covered with layers of cumulus cells.

FIG. 10C shows a cumulus-free MII stage oocyte denuded by manual pipetting.

FIG. 10D shows a cumulus-free MII stage oocyte denuded using the present technology.

FIGS. 11A-11C show images of denudation of a MII stage mouse oocyte using the present technology

FIG. 12 illustrates oocyte preparation comparison between the mechanical procedure and the present technology.

FIG. 13 illustrates embryo development comparison between the mechanical procedure and the present technology.

FIG. 14 shows streamline velocity field experimental data for the method of the present technology.

FIG. 15 illustrates computational fluid dynamic simulations for the methods of the present technology.

### DETAILED DESCRIPTION

The present technology relates to methods and devices for automated microfluidic oocyte denudation.

One aspect of the present technology relates to a microfluidic device for denudation of a cumulus oocyte complex. The device includes a substrate. A first channel having a width of about 200 μm to about 1 mm is located within the substrate. The first channel extends from a first end to a second end of the substrate. The first channel has one or more ridge elements located along a surface thereof. A first port is located in the substrate and in fluid communication with the first end of the channel. A second port is located in the substrate and in fluid communication with the second end of the channel.

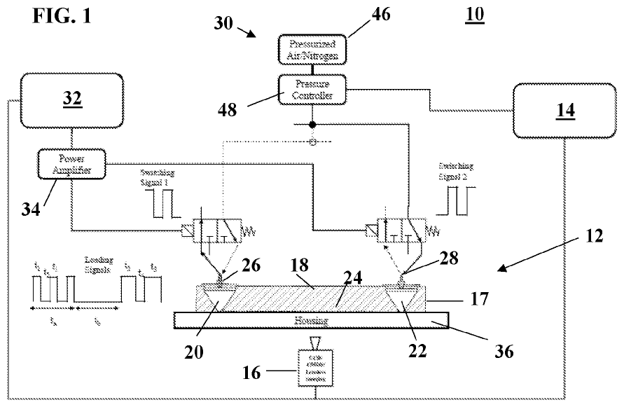

FIG. 1 is a block diagram of environment 10 including a first embodiment of microfluidic device 12 for denudation of a cumulus oocyte complex. Environment 12 includes microfluidic device 12, computing device 14, and imaging device 16, although environment 10 may include other numbers and/or types of elements or devices in other combinations, including additional electronics such as digital to analog converters or additional optical devices, by way of example only.

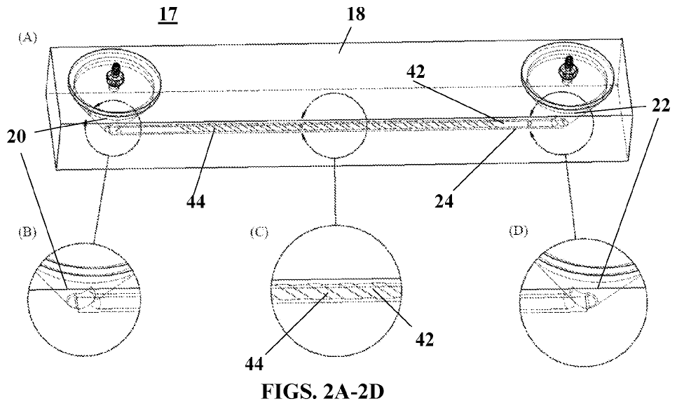

Referring now to FIGS. 1 and 2A-2D, in this example, microfluidic device 12 includes microfluidic chip 17, which includes substrate 18, first port 20, second port 22, and channel 24, as well as first valve 26, second valve 28, pump 30, and controller 32, although microfluidic device 12 may include other types and/or numbers of elements or components in other combinations, such as power amplifier 34, which is coupled to controller 32 and housing 36, which supports substrate 18. In one example, microfluidic device 12 may include only microfluidic chip 17 which is configured to be separately coupled to the additional elements illustrated in FIG. 1. Microfluidic device 12 advantageously provides an integrated, semi-automated device that may be utilized for denudation of a cumulus oocyte complex (COC).

Substrate 18 is configured to house first port 20, second port 22, and channel 24. Substrate 18 may be formed of any suitable biocompatible material, such as glass, polystyrene, polydimethysiloxane (PDMS), or poly(methylacrylate) (PMMA), by way of example only. In this example, substrate 18 is formed as a single layer, although in other examples, as described below, substrate 18 may have multiple layers.

First port 20 and second port 22 are located in substrate 18 and provide openings for the introduction and removal of fluid including COCs from microfluidic chip 17, although substrate 18 may include other numbers of ports in fluid communication with channel 24, as described below. First port 20 and second port 22 may be used as either inlet or outlet ports. In this example, first port 20 and second port 22 are configured as micro-funnels. This configuration advantageously prevents COCs from getting lost during loading into microchip 17. First port 20 and second port 22 extend into substrate 18 and terminate on top of channel 24 as shown in FIGS. 2B and 2D, respectively, such that first port 20 and second port 22 are in direct contact and in fluid communication with channel 24. First port 20 and second port 22 may be sealed using miniaturized soft tube fittings and caps, as shown in FIG. 2A, in order to seal channel 24 during the denudation procedure. In one example, at least a portion of substrate 18 is optically translucent to provide a view of channel 24 for use with imaging device 16, as described in further detail below.

Channel 24 is located within substrate 18 and extends from first end 38, which is coupled to and in fluid communication with first port 20, to second end 40, which is coupled to and in fluid communication with second port 22. Channel 24 has a length between first end 38 and second 40 of about 1 cm to about 10 cm. In embodiments, channel 24 has a length of about 1 cm, 2 cm, 3 cm, 4 cm, 5 cm, 6 cm, 7 cm, 8 cm, 9 cm, or any length in between. Channel 24 includes surface 42 having a number of ridge elements 44 located thereon. In one example, surface 42 is a top surface of channel 24 (based on the orientation of microfluidic chip 17 shown in FIG. 2A, which is the orientation during use of microfluidic device 12), although ridge elements 44 may be located on other surfaces of channel 24. In one example, channel 24 has between 1 to 10 ridge elements 44 per mm along the length of channel 24. In another example, channel 24 has between 2-5 ridge elements 44 per mm along the length of channel 24. In yet another example, channel 24 has 2 ridge elements 44 per mm along the length of channel 24.

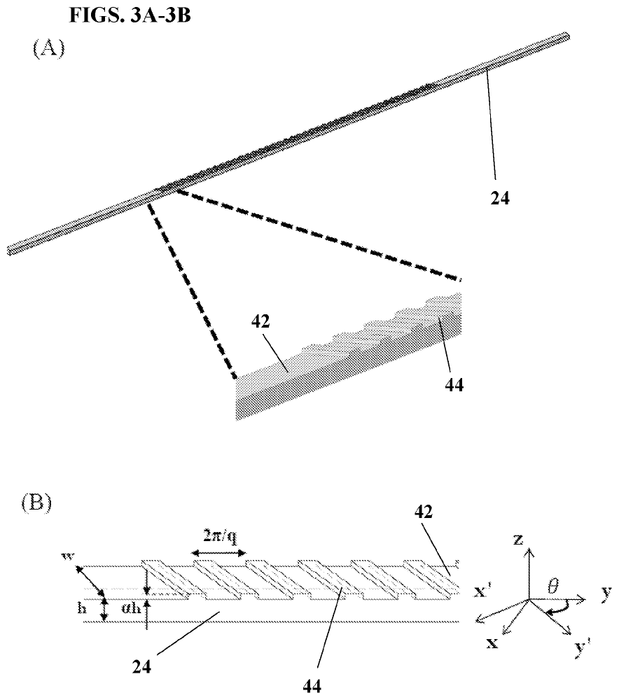

FIGS. 3A and 3 illustrate a perspective view and a schematic view of channel 24 with ridge elements 44 located on surface 42 of channel 24. Although certain aspects will be described with respect to ridge elements 44 as shown in FIGS. 3A and 3B, it is to be understood that other types and/or numbers of ridge elements may be employed in other configurations, as described in further detail below. Ridge elements 44 are configured to generate a secondary flow of fluid within channel 24 when a first flow of fluid is applied between first end 38 and second 40, or vice versa. The secondary flow of fluid causes the first flow to become one of a helical flow, a twisted flow, a vortexed flow, or combinations thereof, which assists in the denudation process. Various configurations of ridge elements may be employed to achieve the desired secondary flow.

Referring again to FIGS. 3A and 3B, the coordinate systems (x, y, z) and (x′, y′, z′) indicate the principle axis orientation of channel 24 and ridge elements 44. In this example, channel 24 extends along the longitudinal axis (y) and ridge elements 44 are oriented along axis (y′). Ridge elements are positioned at an oblique orientation at an oblique angle (θ) with respect to the longitudinal axis (y) of channel 24. In one example, the oblique angle (θ) is less than 90 degrees. In another example, the oblique angle (θ) is in a range from about 30 degrees to about 70 degrees. In yet another example, the oblique angle (θ) is about 45 degrees. In this example, each of ridge elements 44 are positioned in a parallel orientation with respect to the other ridge elements 44 and are equally spaced along channel 24, although other configurations may be employed. Ridge elements 44 are rectangular in shape, although other shapes may be employed such as curvilinear, chevron, offset chevron, or combinations thereof.

Referring now more specifically to FIG. 3B, channel 24 has a width (w) and height (h) that are each between about 200 μm and 1 mm. In embodiments, channel 24 has a width of about 200 μm, 300 μm, 400 μm, 500 μm, 600 μm, 700 μm, 800 μm, 900 μm, or any width in between. Height (h) is sufficient such that a standard size COC may be located within channel 24 and not come into contact with ridge elements 44 during the denudation process. By way of example, standard COC for humans are in the range of 2-3 times the oocyte diameter, which on average is about 110 microns. In another example, mouse oocyte have an average diameter of 90 microns and the COC is 2-3 times bigger. Ridge elements 44 extend across the entire width (w) of channel 24 in this example, although in other examples ridge elements 44 may extend approximately 80-99 percent across the width (w) of channel 24. Ridge elements 44 have a depth (αh) as shown in FIG. 3B that is less than half of the height (h) of channel 24. Ridge elements 44 may have a depth (αh) of between about 100 μm and 500 μm. By way of example only, ridge elements 44 may have a depth (αh) of about 100 μm, 200 μm, 300 μm, 400 μm, 500 μm, or any value therebetween. Ridge elements have a thickness (i.e. the dimension perpendicular to axis y′) that is less than half the width (w) of channel 24. Ridge elements 44 may have a thickness of between about 100 μm and 500 μm. By way of example only, ridge elements 44 may have a depth (αh) of about 100 μm, 200 μm, 300 μm, 400 μm, 500 μm, or any value therebetween. Ridge elements 44 are positioned such that they do not come into a substantial amount of contact with the COC during the denudation process, i.e., ridge elements 44 are not utilized to mechanically denude the oocyte.

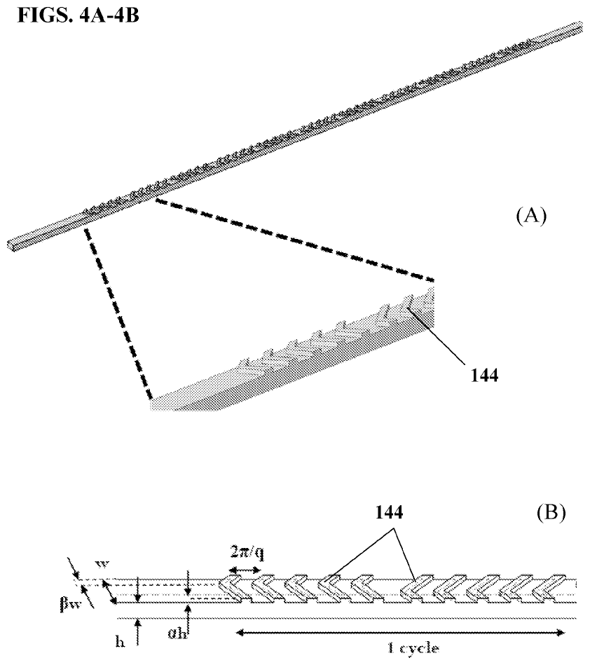

FIGS. 4A and 4B illustrate a perspective view and a schematic view of an alternate embodiment of channel 24 with ridge elements 144 located on surface 42 of channel 24. In this example, ridge elements 144 have a chevron shape, although as described above various shapes may be employed for the ridge elements described herein. Ridge elements 144 are formed in cycles that include two sequential herringbone regions with alternating symmetry with respect to the centerline of channel 24. In this example, the asymmetry vector (β) is a function of the width (w) of channel 24 (0<β<0.3). The asymmetry vector (β) may be constant or may be alternated at each half cycle with respect to the centerline of channel 24. Although this alternate embodiment including ridge elements 144 is described, it is to be understood that numerous other configurations with other shapes and types and/or numbers of ridge elements may be employed along channel 24.

First valve 26 and second valve 28 are coupled to first port 20 and second port 22, respectively. First valve 26 and second valve 28 are high-speed, three-way/two-position solenoid valves, although other types of valves may be employed. In one example, first valve 26 and second valve 28 are biocompatible valves. First valve 26 and second valve 28 are coupled to controller 32 through power amplifier 34, such that the operation of first valve 26 and second valve 28 are controlled by signals from controller 32. By way of example, first valve 26 and second valve 28 may be controlled by rectangular signals generated by controller 32 (e.g., switching signal 1 and switching signal 2 as shown in FIG. 1). First valve 26 and second valve 28 receive signals such that, at each moment, the signal to first valve 26 (switching signal 1) is the inverse of the signal to second valve 28 (switching signal 2) so a complete microfluidic circuit is formed in one direction along channel 24. The flow rate for the fluid inside channel 24 is controlled by controller 32 by adjusting the duty cycle of the generated rectangular signals. For example, a duty cycle of 0% and 100% would active only one of first valve 26 and second valve 28 such that the fluid would flow in channel 24 in a single direction. A duty cycle of 50% results in a zero net fluid flow inside channel 24. Any positive or negative offset from a 50% duty cycle leads to a net fluid in either direction along channel 24.

Pump 30 is in fluid communication with first valve 26 and second valve 28 such that pump 30 is configured to provide pressure within channel 24 to generate a fluid flow along channel 24 based on the position of first valve 26 and second valve 28, as described above. In one example, pump 30 is a pneumatic pump. Pump 30 includes a pressure source 46 and a pressure controller 48 to control the amount of pressure provided to first valve 26 and second valve 28 to control the rate of fluid flow in channel 24. Pressure controller 48 is coupled to computing device 18 and may receive instructions therefrom to alter the amount of pressure provided to first valve 26 and second valve 28.

Controller 32 may be any suitable device, such as a microcontroller, for providing signals, such as the rectangular switching signals shown in FIG. 1, to first valve 26 and second valve 28. By way of example only, controller 32 could be model number LHDA2431115H from Lee Company (Westbrook, Conn.). Controller 32 is coupled to computing device 18 by a communication network and may receive one or more instructions from computing device 18 to alter the duty cycle of first valve 26 and second valve 28, as described above.

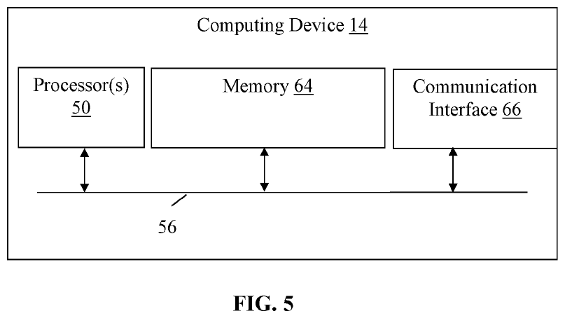

Referring now more specifically to FIGS. 1 and 5, computing device 14 includes one or more processor(s) 50, memory 52, and communication interface 54 that are coupled together by a bus 56 or other communication link, although computing device 14 can include other types and/or numbers of elements in other configurations.

Processor(s) 50 of computing device 14 may execute programmed instructions stored in memory 52 for any number of the functions or other operations illustrated and described by way of the examples herein, including determining, based on images from imaging device 16, a state of denudation of a COC in channel 24 (as defined below) and providing instructions to controller 32 to change the duty cycle to alternately open and/or close first valve 26 and second valve 28, by way of example only. Processor(s) 50 of computing device 14 may include one or more graphic processing units (GPUs), CPUs, or general processors with one or more processing cores, for example, although other types of processor(s) can be used.

Memory 52 of computing device 14 stores the programmed instructions for one or more aspects of the present technology as illustrated and described herein, although some or all of the programmed instructions could be stored elsewhere. A variety of different types of memory storage devices, such as random access memory (RAM), read only memory (ROM), hard disk drive (HDD), solid state drives (SSD), flash memory, or other computer readable medium that is read from and written to by a magnetic, optical, or other reading and writing system that is coupled to processor(s) 50 can be used for memory 52.

Accordingly, memory 52 of computing device 14 can store application(s) that can include executable instructions that, when executed by computing device 14, cause computing device 14 to perform actions, such as to perform methods for denudation of a COC as illustrated and described by way of the examples herein. The application(s) can be implemented as modules or components of other application(s). Further, the application(s) can be implemented as operating system extensions, modules, plugins, or the like.

Communication interface 54 of computing device 14 operatively couples and allows for communication between computing device 14, imaging device 16, controller 32, and pressure controller 48, which are all coupled together by one or more communication network(s), although other types and/or numbers of connections and/or configurations to other device and/or elements can be used. Communication network(s) can include any number and/or types of communication networks, such as local area network(s) (LAN(s)) or wide area network(s) (WAN(s)), and/or wireless networks, although other types and/or number of protocols and/or communication network(s) can be used.

Although embodiments of computing device 14 are described and illustrated herein, computing device 14 can be implemented on any suitable computing system or computing device. It is to be understood that the devices and systems described herein are bar exemplary purposes and many variations of the specific hardware and software are possible, as will be appreciated by those skilled in the relevant art(s).

Alternatively, each of the systems may be conveniently implemented using one or more general purpose computer systems, microprocessors, digital signal processors, and micro-controllers, programmed according to the teachings described and illustrated herein. In addition, two or more computing systems of devices can be substituted for any one of the systems described above. Accordingly, principles and advantages of distributed processing, such as redundancy and replication, also can be implemented, as desired, to increase the robustness and performance of the devices and systems described above. The embodiments of the present application may also be implemented on a computer system or systems that extend across any suitable network using any suitable interface mechanisms and communications technologies, including, by way of example only, telecommunications in any suitable form (e.g., voice and modem), wireless communications media, wireless communications networks, cellular communications networks, G3 communications networks, Public Switched Telephone Networks (PSTNs), Packet Data Networks (PDNs), the Internet, intranets, and combinations thereof.

Imaging device 16 may be any suitable optical imaging device to obtain images channel 24, or more specifically, a COC located within channel 24, although other appropriate imaging devices may be employed. By way of example only, imaging device 16 may be a charge coupled device (CCD) camera, or a complementary metal-oxide-semiconductor (CMOS) camera, although any suitable imaging device may be employed. Imaging device 16 is coupled to computing device 14 to provide images of at least a portion of channel 24 (including a COC located therein) in accordance with the methods disclosed herein.

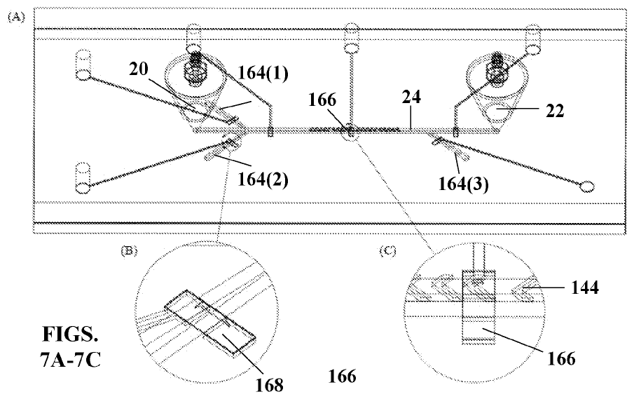

FIG. 7 is a block diagram of environment 100 including a second embodiment of microfluidic device 112 for denudation of a cumulus oocyte complex. Environment 100 includes the same elements as environment 10 as described above, except as described below. Environment 100 includes microfluidic device 112 as well as additional pressure controller 148 used to provide pressure to additional valves on microfluidic device 112 as described below.

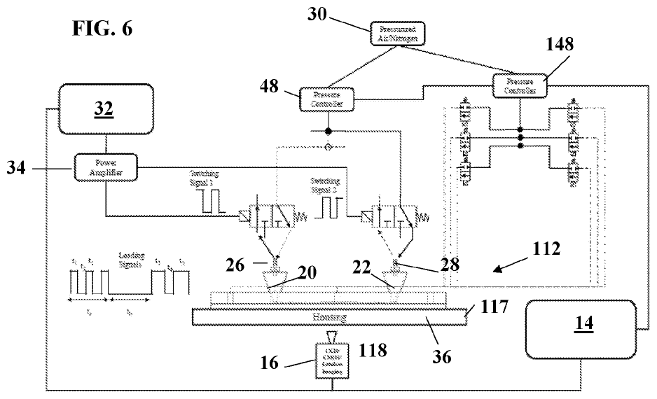

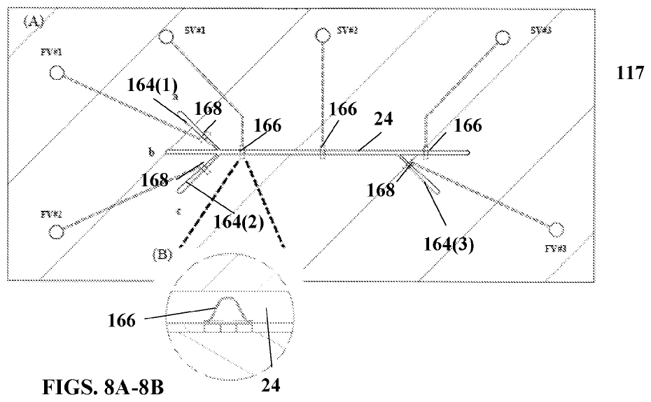

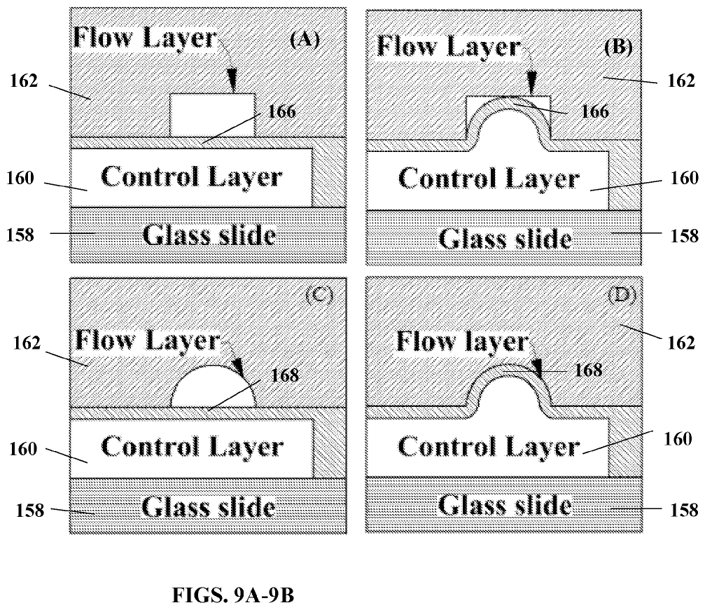

Referring now to FIGS. 6-9, microfluidic device 112 is the same in structure and operation as microfluidic device 12 except as detailed below. Microfluidic device 112 includes microfluidic chip 117, which includes substrate 118 that is constructed of multiple layers as shown in FIGS. 9A-9D. In this example, microfluidic chip 117 includes base layer 158, control layer 160, and flow layer 162. Base layer 158 is formed from a glass material. Control layer 160 is formed on top of base layer 158 and is constructed of a reversibly deformable material, such as polydimethylsiloxane (PDMS). Control layer 160 provides a number of additional valves for microfluidic chip 117 as described below as control layer 160 may be deformed into flow layer 162. By way of example, control layer 160 provides sieve valves 166 located along channel 24. Sieve valves 166 may be employed to provide constriction in channel 24 to provide additional pressure control within channel 24. FIGS. 9A and 9B illustrate sieve valve 166 before (FIG. 9A) and after (FIG. 9B) pressurization.

Flow layer 162 includes channel 24 as well as supplementary channels 164(1)-164(3) in fluid communication with channel 24. Supplementary channels 164(1)-164(3) are each associated with ports (not shown) for the introduction and/or removal of material from microfluidic chip 117. Further, supplementary channels 164(1)-164(3) may be associated with additional solenoid valves (not shown) operated by pressure controller 148. By way of example, supplementary channel 164(1) may provide a channel for introducing HA to the channel 24 to assist in the denudation process, supplementary channel 164 may provide a channel for introducing a washing media, such as a buffer solution, such as embryomax M2 medium with phenol red and hyaluronidase (M2+HA), product number MR-051 from Sigma-Adrich (Darmstadt, Germany) to channel 24, while supplementary channel 164(3) may be used for removal of materials from microfluidic chip 117, although supplementary channels 164(1)-164(3) may be used for the introduction of any other types and/or number of fluids to channel 24. Control layer 160 also provides full valves 168 that may be used to open and close access from the ports associated with supplementary channels 164(1)-164(3) to channel 24. FIGS. 9C and 9D illustrate full valve 168 before (FIG. 9C) and after (FIG. 9D) pressurization. As shown in FIG. 9D, control layer 160 blocks flow layer 162 in the fully pressurized state to preclude fluid from passing full valve 168.

Another aspect of the present technology relates to a system for denudation of a cumulus oocyte complex. The system includes the microfluidic device of the present technology. An optical imaging device is configured to image a portion of the channel including a cumulus oocyte complex of the microfluidic device. A computing device is coupled to the optical imaging device. The computing device includes a processor coupled to a memory and configured to execute programmed instructions stored in the memory including determining, based on one or more images received from the optical imaging device, a state of denudation of the cumulus oocyte complex located in the portion of the channel. One or more instructions are provided to the controller to alternately open and close the first valve and the second valve.

Yet another aspect of the present technology relates to a method for denudation of a cumulus oocyte complex. The method includes providing the microfluidic device of the present technology. A fluid including a cumulus oocyte complex is introduced into the channel of the microfluidic device through the first port. The first valve and the second valve are activated such that the cumulus oocyte complex is translated along the channel in a first direction toward the second end from the first end along the one or more ridge elements.

An exemplary method for denudation of a cumulus oocyte complex (COC) will now be described with respect to FIGS. 1-3B. The method may be similarly employed with the other embodiments disclosed herein. First, a fluid including a COC is introduced into channel 24 of microfluidic chip 17 through first port 20, although the fluid including the COC could alternatively be introduced to channel 24 through second port 22. First port 20 is configured as a funnel to ensure that the COC is not lost during introduction to channel 24. In example, the COC is introduced into channel 24 using a pulsed flow using first valve 26 and second valve 28.

Next, first valve 26 and the second valve 28 are activated by altering the duty cycle as described above to generate fluid flow in channel 24 such that the COC is translated along channel 24 in a first direction toward second end 40 from first end 38 along ridge elements 44. Ridge elements 44 generate a secondary flow of fluid within channel 24 when the first flow of fluid is applied between first end 38 and second 40, or vice versa. The secondary flow of fluid causes the first flow to become one of a helical flow, a twisted flow, a vortexed flow, or combinations thereof, which assists in the denudation process. The secondary flow generates vortex forces that are utilized for denudation of the COC. In some examples, denudation is performed almost entirely by the vortex forces generated from the secondary flow. In some examples, denudation occurs without any mechanical contact with ridge elements 44, although there may be some incidental contact with ridge elements 44 in other examples. Denudation does not require the COC to contact ridge elements 44, although some incidental contact may occur. The denudation does not require additional mechanical contact with ridge elements 44. The duty cycle of first valve 26 and second valve 28 may be altered to oscillate the fluid flow, and the COC, in both directions along channel 24. Oscillation is introduced in microfluidic channel 24 in order to achieve an infinite length that is necessary to achieve submicron particle focusing. By way of example, the COC can be translocated along channel 24 at a rate of between 1000 μm/second to 10000 μm/second.

Next, oscillation of the fluid back and forth is performed until cumulus cells are separated from the COC to produce a denuded oocyte based on the fluid flow in channel 24 using primarily the secondary flow generated by ridge elements 44. Specifically, as the fluid is oscillated back and forth the fluid passes ridge elements 44. As the fluid oscillates, the secondary flow is generated, which produces vortex forces to provide denudation as described above. Oscillation simply allows for the length of channel 24 to be shortened. The denuded oocyte may then be removed from microfluidic chip 17, for example through second port 22.

A further aspect of the present technology relates to a method for denudation of a cumulus oocyte complex. The method includes providing the system of the present technology. A fluid including a cumulus oocyte complex is introduced into the channel of the microfluidic device through the first port. The first valve and the second valve are activated such that the cumulus oocyte complex is translated along the channel in a first direction toward the second end from the first end along the one or more ridge elements. The position and/or state of the cumulus oocyte complex is monitored using the optical imaging device. The activation of the first valve and the second valve is adjusted based on the position and/or state of the cumulus oocyte complex.

In one example, imaging device 16 may be used to image a portion of channel 24 including the COC. The images from imaging device 16 are provided to computing device 14. Computing device 14 analyzes the images to monitor the position and/or state of COC within channel 24. The state of the COC indicates the extent of denudation of the oocyte, i.e., whether the oocyte is partially denuded or completely denuded. Computing device 14 may then alter the duty cycle of first valve 26 and second valve 28, or the pressure provided by pressure controller 48, to alter the fluid flow in channel 24 to impact the denudation process.

### EXAMPLES

The examples below are intended to exemplify the practice of embodiments of the disclosure but are by no means intended to limit the scope thereof.

### Example 1

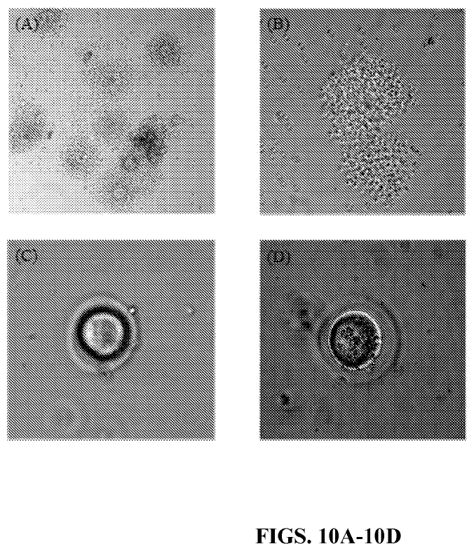

Studies were conducted using the devices and methods described above compared to conventional methods. A cohort of oocytes were retrieved from super-ovulated B6D2F1 adult mice as illustrated in FIG. 10A. FIG. 10B illustrates a metaphase II stage (MII) stage mouse oocyte covered with layers of cumulus cells. The oocytes were exposed to 1 mg/ml HA for 30 second and immediately rinsed and loaded into the microfluidic device. Results for the microfluidic chip and conventional methods were comparable. FIGS. 10C and 10D illustrate a cumulus-free MII stage oocyte denuded by manual pipetting and a cumulus-free MII stage oocyte denuded using the present technology, respectively.

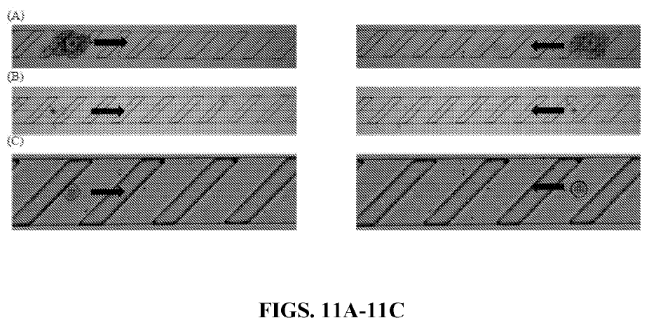

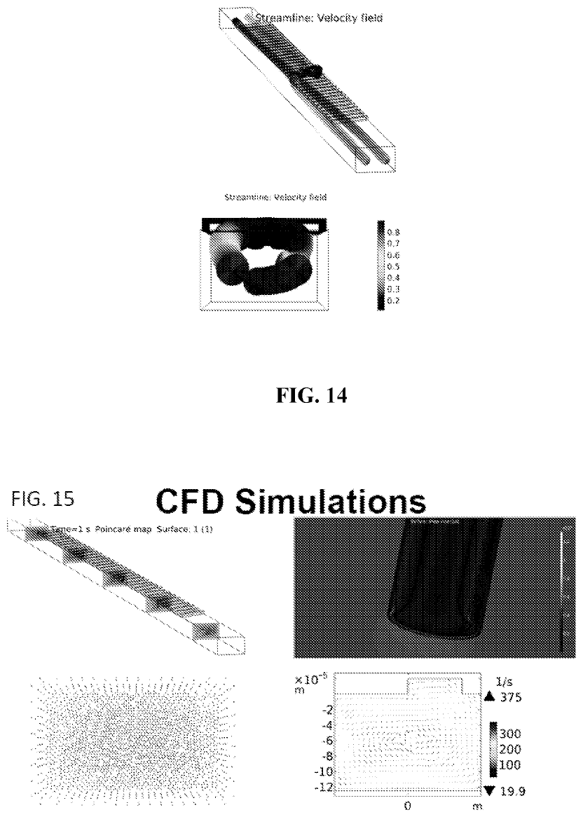

FIGS. 11A-11C show images of denudation of a MII stage mouse oocyte using the present technology, which relies primarily on the second flow generated by ridge elements in the channel, as opposed to mechanical denudation. An alternating flow pressure was set to 2 mbar at a frequency of 5 Hz. FIG. 11A illustrates the freshly loaded COC without HA treatment. FIG. 11B shows the COC in a partially denuded state. FIG. 11B shows a fully denuded COC after 30 seconds of treatment. FIGS. 14 and 15 show streamline velocity field experimental data and computational fluid dynamic (CFD) simulations for the methods of the present technology that illustrate the generation of a secondary flow within the channel that is employed in the denudation process.

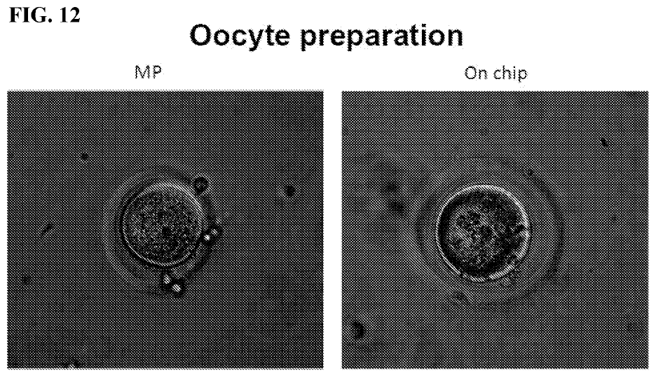

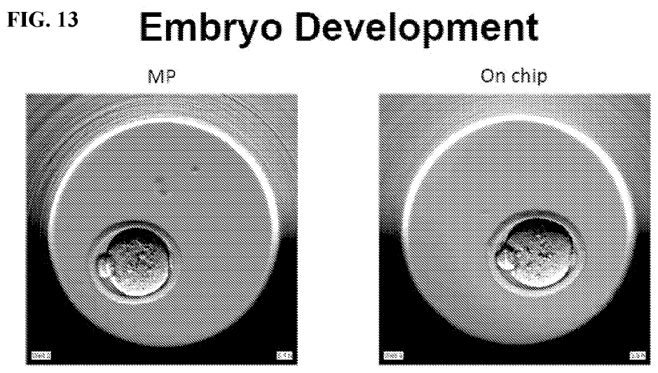

To ensure that the disclosed devices and methods do not risk developmental potential of the gametes, piezo-actuated intracytoplastic sperm injection (IC SI) were performed on 50 oocytes processed by each method. Oocytes denuded by either mechanical procedures or the devices and systems of the present technology showed similar cleavage parameters. FIG. 12 illustrates the oocyte preparation comparison between the mechanical procedure and the present technology, while FIG. 13 illustrates embryo development comparison between the mechanical procedure and the present technology.

Results of the comparison between the conventional techniques (left column) and the devices and methods of the present technology (right column) are illustrated in Table 1.

| | Embryology Outcomes (%) | | |
|---|---|---|---|
| | | Disclosed | |
| | Conventional | microfluidic | P |
| f | (n = 50) | (n = 50) | value |
| CC | 91.0 | 95.0 | NS |
| deprivation | | | |
| ICSI survival | 82.0 | 84.0 | NS |
| Fertilization | 90.2 | 92.9 | NS |
| Blastocyst | 87.8 | 90.5 | NS |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A microfluidic device for denudation of a cumulus oocyte complex, the device comprising:
a substrate;
a first channel having a width of about 200 μm to about 1 mm located within the substrate and extending from a first end to a second end, the first channel having a one or more ridge elements located along a surface thereof;
a first port located in the substrate and in fluid communication with the first end of the channel; and
a second port located in the substrate and in fluid communication with the second end of the channel.

2. The microfluidic device of claim 1, wherein the one or more ridge elements are located along a top surface of the channel as oriented during use of the microfluidic device.

3. The microfluidic device of claim 1, wherein the one or more ridge elements are configured to generate a secondary flow of fluid within the channel when a first flow of fluid is applied from the first end of the channel to the second end of the channel.

4. The microfluidic device of claim 3, wherein the secondary flow of fluid causes the first flow of fluid to become one of a helical flow, a twisted flow, a vortexed flow, or combinations thereof.

5. The microfluidic device of claim 1, wherein the one or more ridge elements are positioned in an oblique orientation with respect to a longitudinal axis of the channel.

6. The microfluidic device of claim 5, wherein the one or more ridge elements are positioned at an oblique angle of less than 90 degrees with respect to the longitudinal axis of the channel.

7. The microfluidic device of claim 6, wherein the oblique angle is in a range from about 30 degrees to about 70 degrees.

8. The microfluidic device of claim 7, wherein the oblique angle is about 45 degrees.

9. The microfluidic device of claim 1, wherein each of the one or more ridge elements are positioned in a parallel orientation with respect to each of the other one or more ridge elements.

10. The microfluidic device of claim 1, wherein the one or more ridge elements are equally spaced along the channel.

11. The microfluidic device of claim 1, wherein the one or more ridge elements extend an entire width of the channel.

12. The microfluidic device of claim 1, wherein the one or more ridge elements are curvilinear.

13. The microfluidic device of claim 1, wherein the one or more ridge elements are one of rectangular, chevron, offset chevron, or combinations thereof.

14. The microfluidic device of claim 1, wherein the channel comprises a length of about 1 cm to about 10 cm.

15. The microfluidic device of claim 1, wherein the one or more ridge elements have a depth in the surface of the substrate of less than half of a height of the channel.

16. The microfluidic device of claim 15, wherein one or more ridge elements have a depth in surface of the substrate from about 100 μm to about 500 μm.

17. The microfluidic device of claim 1, wherein the one or more ridge elements have a thickness of less than half a width of the channel.

18. The microfluidic device of claim 17, wherein the one or more ridge elements have a thickness of about 100 μm to about 500 μm.

19. The microfluidic device of claim 1, wherein the channel comprises 1 to 10 of the one or more ridge elements per mm.

20. The microfluidic device of claim 19, wherein the channel comprises 2-5 ridges per mm.

21. The microfluidic device of claim 20, wherein the channel comprises 2 ridges per mm.

22. The microfluidic device of claim 1, wherein at least a portion of the substrate is optically translucent to provide a view of the channel.

23. The microfluidic device of claim 1 further comprising one or more sieve valves located along the channel.

24. The microfluidic device of claim 1 further comprising at least one secondary channel in fluid communication with the channel.

25. The microfluidic device of claim 24 further comprising a third port located in the substrate and in fluid communication with at least one secondary channel.

26. The microfluidic device of claim 1, wherein the substrate comprises a base layer, a control layer, and a flow layer.

27. The microfluidic device of claim 26, wherein the base layer is a glass material.

28. The microfluidic device of claim 26, wherein the control layer is a reversibly deformable material.

29. The microfluidic device of claim 26, wherein the channel is located in the flow layer of the substrate.

30. The microfluidic device of any one of the preceding claims further comprising:
a first valve coupled to the first port;
a second valve coupled to the second port;
a pump in fluid communication with the first valve and the second valve; and
a controller coupled to the pump and configured to alternately open and close the first valve and the second valve.

31. The microfluidic device of claim 30, wherein the first valve and the second valve are three-way valves.

32. The microfluidic device of claim 30, wherein the pump is a pneumatic pump.

33. A system for denudation of a cumulus oocyte complex, the system comprising:
the microfluidic device of claim 30;
an optical imaging device configured to image a portion of the channel including a cumulus oocyte complex of the microfluidic device; and
a computing device coupled to the optical imaging device, the computing device comprising a processor coupled to a memory and configured to execute programmed instructions stored in the memory comprising:
determining, based on one or more images received from the optical imaging device, a state of denudation of the cumulus oocyte complex located in the portion of the channel; and
provide one or more instructions to the controller to alternately open and close the first valve and the second valve.

34. A method for denudation of a cumulus oocyte complex, the method comprising:
providing the microfluidic device of claim 30;
introducing a fluid into the channel of the microfluidic device through the first port, the fluid comprising a cumulus oocyte complex; and
activating the first valve and the second valve such the cumulus oocyte complex is translated along the channel in a first direction toward the second end from the first end along the one or more ridge elements.

35. The method of claim 34, wherein the activating is performed to alternately translate the cumulus oocyte complex along the channel in the first direction toward the second end from the first end and in a second direction toward the first end from the second end along the one or more ridge elements.

36. The method of claim 35, wherein the activating is performed until cumulus cells are separated from the cumulus oocyte complex to produce a denuded oocyte.

37. The method of claim 36 further comprising:
removing the denuded oocyte from the microfluidic device.

38. The method of claim 34, wherein introducing the fluid comprises providing a pulsed flow of fluid to the channel.

39. A method for denudation of a cumulus oocyte complex, the method comprising:
providing the system of claim 30;
introducing a fluid into the channel of the microfluidic device through the first port, the fluid comprising a cumulus oocyte complex;
activating the first valve and the second valve using the pump such that the cumulus oocyte complex is translated along the channel in a first direction toward the second end from the first end along the one or more ridge elements;
monitoring the position and/or state of the cumulus oocyte complex using the optical imaging device; and
adjusting, by the computing device, the activation of the first valve and the second valve based on the position and/or state of the cumulus oocyte complex.

40. The method of claim 39, wherein the activating is performed to alternately translate the cumulus oocyte complex along the channel in the first direction toward the second end from the first end and in a second direction toward the first end from the second end along the one or more ridge elements.

41. The method of claim 40, wherein the activating is performed until cumulus cells are separated from the cumulus oocyte complex to produce a denuded oocyte.

42. The method of claim 41 further comprising:
removing the denuded oocyte from the microfluidic device.

43. The method of claim 40, wherein the cumulus oocyte complex is alternately translated along the channel at a rate of between about 1000 μm/second to about 10000 μm/second.
